(19) United States
(12) Patent Application Publication
(10) Pub. No.: US 2025/0366883 A1
(43) Pub. Date: Dec. 4, 2025

(54) Image-Guided Instrument Positioning System

(71) Applicant: Georgia Tech Research Corporation, Atlanta, GA (US)

(72) Inventors: Yue Chen, Atlanta, GA (US); Zhefeng Huang, Atlanta, GA (US)

(21) Appl. No.: 19/220,986

(22) Filed: May 28, 2025

Related U.S. Application Data
(60) Provisional application No. 63/652,505, filed on May 28, 2024.

(51) Int. Cl. A61B 17/34; A61B 5/00; A61B 10/00; A61B 10/02; A61B 17/00; A61B 34/20

(52) U.S. Cl. CPC A61B 17/3403; A61B 5/4312; A61B 10/0041; A61B 10/0233; A61B 34/20; A61B 2017/00039; A61B 2017/00544; A61B 2017/3409; A61B 2017/3411; A61B 2034/2051

(57) ABSTRACT

A system for accurately positioning a medical instrument is provided. The system includes a guide template for directing the medical instrument and an automated chassis for moving the guide template. The guide template has a body defining a curved channel extending through the guide template. The curved channel is configured to receive the medical instrument. The automated chassis includes an actuator and a rail system. The guide template is coupled to the rail system of the automated chassis. A position of the guide template is movable along a length of the rail system. The actuator moves the guide template along a length of the rail system. The system facilitates positioning the medical instrument relative to a target tissue under real-time guidance.

### CROSS-REFERENCE TO RELATED APPLICATIONS

This application claims the benefit of U.S. Provisional Application No. 63/652,505, filed May 28, 2024, which is incorporated by reference in its entirety for all purposes.

### BACKGROUND

Magnetic Resonance Imaging (MRI) is an excellent imaging modality for breast biopsy with high soft tissue contrast, ability to detect small and occult lesions, and absence of ionization radiation. It is considered the gold standard for breast cancer screening and the American Cancer Society recommends all women with high risk factors to obtain MRI scans due to its high sensitivity. However, current screening techniques suffer at least in part because MRI imaging yields low specificity, meaning that definitive diagnosis requires biopsy using an instrument such as a biopsy needle. Furthermore, current biopsy techniques result in high false negative rates due to incorrect placement of the biopsy needle. Such false negatives arising from incorrect biopsy needle placement can cause patients to miss opportunities for treatment. Of the 176,000 breast biopsies performed under MRI guidance each year in the United States, 25,000 result in false negatives. Some reasons for this error rate include: (1) lack of real-time imaging; (2) soft tissue deformation during biopsy needle insertion; and (3) low resolution and accuracy of current biopsy needle placement methods. As a result, patients may receive late cancer treatment, leading to poor prognoses and deaths that could have been prevented by superior diagnosis methods and earlier initiation of treatment. Furthermore, time is often of the essence during biopsy procedures because contrast agents used to visualize the suspected legion rapidly dissipate. In typical instances, a surgeon must commit to memory the location of the lesion and “guess” the correct location to target with the biopsy needle. If the lesion could be seen in real-time during insertion of the biopsy needle, targeting accuracy could be improved. Similarly, other needle-based interventions, such as those utilizing microwave ablation devices, are similarly hindered by challenges with needle placement. Accordingly, the state-of-the-art magnetic resonance-based interventions involve suboptimal workflow due to an inability to accurately place medical instruments (such as biopsy needles) under magnetic resonance guidance.

Therefore, a need exists for improved targeting of tissues such as suspected lesions in the course of magnetic resonance-guided interventions.

### SUMMARY

Provided herein are systems, devices, and methods for positioning a medical instrument in a patient. In some aspects, the disclosed systems, devices, and methods are compatible with intraoperative MRI, thereby enabling users to accurately position a medical instrument relative to a target tissue using real-time MRI feedback.

As a non-limiting example, in some implementations, the present disclosure can include systems and devices that can accurately place a needle into a patient in response to user input. The systems can include a guide template for the needle (or any other medical instrument). The user can optionally monitor the position of the needle using a magnetic resonance imaging (MRI) system to locate where the needle is in the patient and use the systems and devices of the present disclosure to guide or move the needle to a desired position inside the patient.

In one aspect, the techniques described herein relate to a system for positioning a medical instrument. The system includes a guide template and an automated chassis. The guide template has a body defining a curved channel extending through the guide template. The curved channel is configured to receive the medical instrument. The automated chassis includes an actuator and a rail system. The guide template is coupled to the rail system of the automated chassis. A position of the guide template is movable along a length of the rail system. The actuator moves the guide template along a length of the rail system.

In various implementations, the system further includes a control system operatively coupled to the actuator. The actuator receives an output from the control system to determine a target position of the guide template along the length of the rail system.

In various implementations, the body of the guide template includes a first face, a second face opposite the first face, a third face, and a fourth face opposite the third face. In various implementations, the first face of the guide template defines a first opening and the third face of the guide template defines a second opening, and wherein the curved channel extends through the body in a curved direction between the first opening and the second opening.

In various implementations, the medical instrument includes a proximal end and a distal end and is configured to slidably move within the curved channel.

In various implementations, the fourth face of the guide template includes coupling means for coupling the guide template to the rail system.

In various implementations, the guide template defines a plurality of additional curved channels configured to receive the medical instrument. The curved channel and plurality of additional curved channels together define a matrix of curved channels.

In various implementations, a first set of curved channels of the matrix are aligned across a width of the third face of the guide template. In various implementations, a second set of curved channels of the matrix are aligned across a width of the third face of the guide template and are disposed directly adjacent the first set of curved channels along a length of the third face.

In various implementations, the actuator includes a magnetic resonance compatible actuator. In various implementations, the actuator is a pneumatic actuator.

In various implementations, a distal end of the medical instrument includes a micro-tracking coil configured to be located using a magnetic resonance imaging (MRI) machine. In various implementations, the system further includes a magnetic resonance imaging (MRI) machine configured to continuously image the medical instrument. In various implementations, the system further includes a display operably coupled to the MRI machine and configured to display the position of the medical instrument to the user. In various implementations, the system further includes a processing unit operatively coupled to the MRI machine, wherein the processor is configured to receive images from the MRI machine and provide a corresponding output to the control system, and wherein the control system is configured to move the guide template toward the target position.

In various implementations, the system further includes a breast coil. In various implementations, the target position of the guide template corresponds to the distal end of the medical instrument being positioned within the breast coil.

In various implementations, the medical instrument includes a needle. In various implementations, the needle is a biopsy needle.

In one aspect, the techniques described herein relate to a method of positioning a medical instrument in a patient. The method includes: receiving images from a magnetic resonance imaging (MRI) machine; moving a guide template along a length of a rail system of an automated chassis toward a target position adjacent the patient, wherein the guide template is coupled to a rail system of an automated chassis; and advancing a medical instrument through a curved channel defined in the guide template.

It should be understood that the examples described herein are only non-limiting examples and that embodiments of the present disclosure can be used for a variety of measurement techniques.

Additional advantages of the disclosed systems and methods will be set forth in part in the description which follows and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings and from the claims.

Throughout the description and claims of this specification, the word “comprise” and other forms of the word, such as “comprising” and “comprises,” means including but not limited to, and is not intended to exclude, for example, other additives, components, integers, or steps.

### BRIEF DESCRIPTION OF THE DRAWINGS

FIG. 1 shows a perspective view of a guide template for directing a medical instrument toward a target tissue, in accordance with an illustrative embodiment.

FIG. 2 shows a perspective view of a system for positioning a medical instrument. The system includes the guide template of FIG. 1, illustrated in partial transparency, as well as an automated chassis for moving the guide template, in accordance with an illustrative embodiment.

FIG. 3 shows a diagram of a control system in use with an example system for positioning a medical instrument, in accordance with an illustrative embodiment.

FIG. 4 shows a top view of the example system of FIG. 2 in use with a medical instrument, in accordance with an illustrative embodiment.

FIG. 5 shows a front view of the example system of FIG. 2 positioned relative to a patient, in accordance with an illustrative embodiment.

FIG. 6 shows a top view of the example system of FIG. 2 in use with a medical instrument and positioned relative to a patient, in accordance with an illustrative embodiment. The guide template is illustrated in partial transparency.

FIG. 7 shows a perspective view of the example system of FIG. 2 in use with a medical instrument and positioned relative to a patient, in accordance with an illustrative embodiment. The guide template is illustrated in partial transparency.

FIG. 8 shows a side view of the example system of FIG. 2 positioned relative to a patient, in accordance with an illustrative embodiment.

FIG. 9 shows a back view of the example system of FIG. 2 positioned relative to a patient, in accordance with an illustrative embodiment. The guide template is illustrated in partial transparency.

FIG. 10 shows a bottom view of the example system of FIG. 2 positioned relative to a patient, in accordance with an illustrative embodiment. The guide template is illustrated in partial transparency.

FIG. 11 shows a bottom perspective view of the example system of FIG. 2 positioned relative to a patient, in accordance with an illustrative embodiment. The guide template is illustrated in partial transparency.

FIG. 12 shows a diagram of a computing system for use with an example system for positioning a medical instrument, in accordance with an illustrative embodiment.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

To facilitate an understanding of the principles and features of various embodiments of the present invention, they are explained hereinafter with reference to their implementation in illustrative embodiments.

In one aspect, provided herein is a system for positioning a medical instrument relative to a target tissue in a patient. Although various examples described and illustrated herein describe the system in the context of a breast biopsy, in which the medical instrument is a breast biopsy needle and the target tissue is a suspected cancerous lesion in the breast of a patient, these are non-limiting examples. Accordingly, it is contemplated that the systems, methods, and devices provided herein can be used to place any medical instrument relative to any target tissue. Furthermore, the systems, methods, and devices can be implemented in surgical procedures other than needle biopsies. For example, the systems and devices described herein can be used to position ablation instruments or to supplement other minimally invasive procedures, endoscopic, laparoscopic, and/or robot surgery. Furthermore, although the systems and devices provided are compatible with use in conjunction with magnetic resonance imaging, it is contemplated herein that the systems, methods, and devices described can be implemented in the conjunction with other imaging modalities.

### Example System

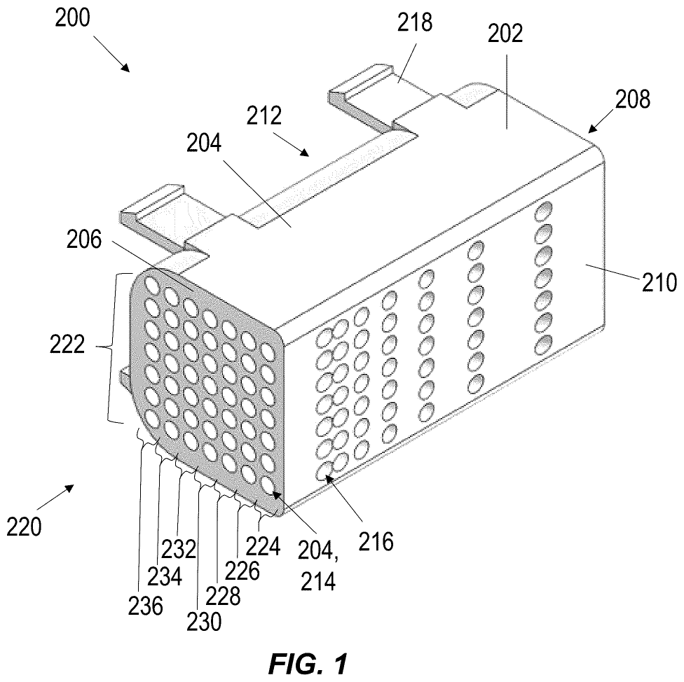

FIG. 1 shows a guide template 200 for directing a medical instrument (for example, medical instrument 150) toward a target tissue, in accordance with one implementation. As shown in the example of FIG. 1, the guide template 200 includes a body 202 that defines a curved channel 204 extending therethrough. As shown in FIG. 1, the body 202 of the guide template 200 generally includes a first face 206, a second face 208 on a side of the body 202 opposite the first face 206, a third face 210, and a fourth face 212 on a side of the body 202 opposite the third face 210. In use, the third face 210 of the guide template 200 generally faces toward the patient.

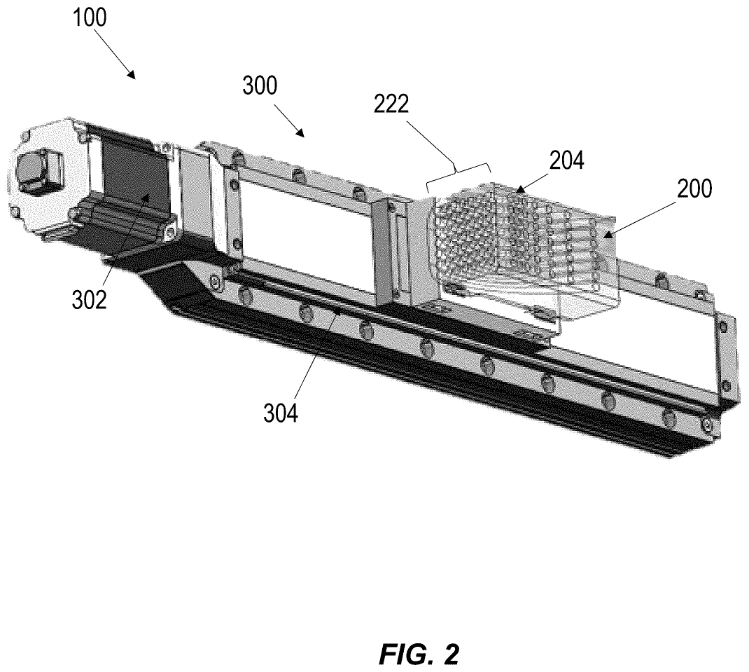

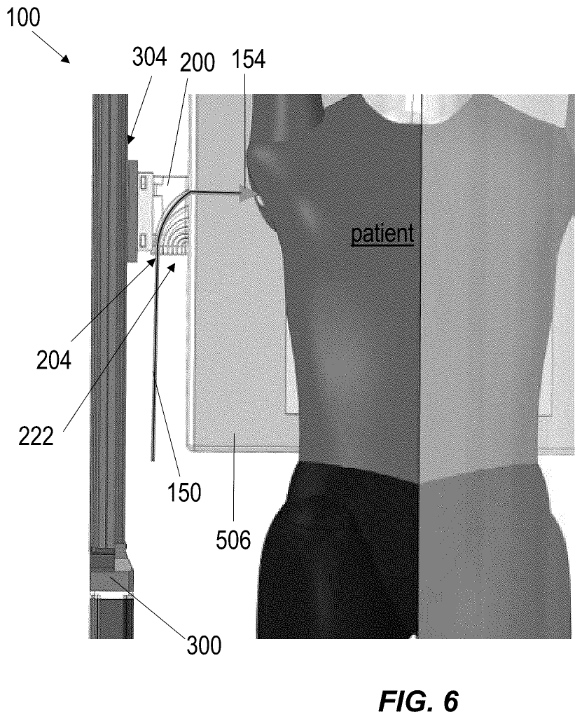

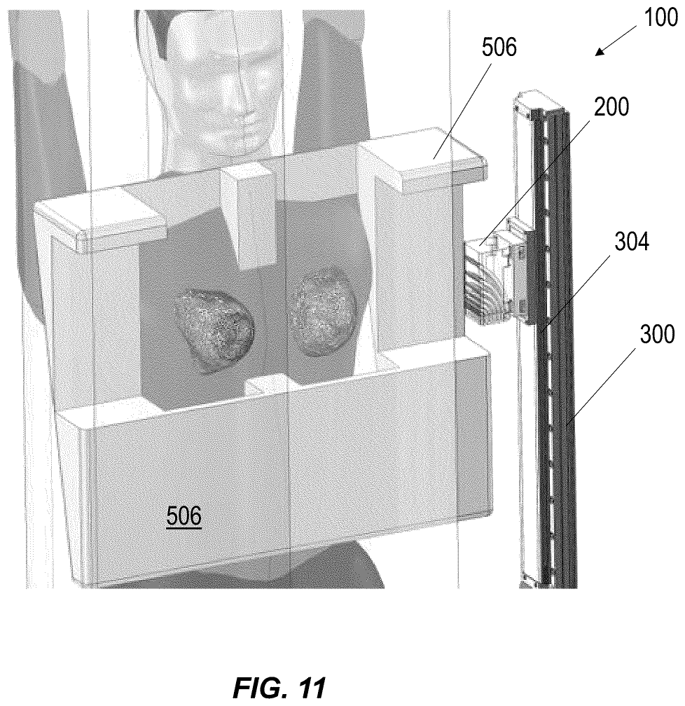

In the illustrated implementation, the curved channel 204 extends between the first face 206 and the third face 210. As further provided herein, the curved channel 204 is sized and configured to receive the medical instrument 150. Specifically, the curved channel 204 extends through the body 202 in a curved direction between a first opening 214 defined in the first face 206 and a second opening 216 defined in the third face 210. Advantageously, the curved path of the curved channel 204 allows for redirection of a medical instrument 150 advanced through the curved channel 204 of the guide template 200. Specifically, a medical instrument 150 can be fed into the first opening 214 from a first direction that would not intersect target tissue of the patient and then be diverted by the curved channel 204 toward a second direction that does intersect the patient and is positioned to facilitate advancement of the medical instrument 150 to the target tissue. Example implementations of the curved channel 204 are made visible provided in FIG. 2, FIG. 6, and FIG. 11, each of which show illustrative examples of the guide template 200 in partial transparency.

Of course, it is contemplated herein that the curved channel 204 can extend between other faces of the guide template 200, so long as the curved channel 204 is curved so as to redirect a medical instrument 150 fed into the first opening 214 through the body 202 and out the second opening 216 toward the patient.

As provided herein, the guide template 200 can define more than one curved channel 204. For instance, the example guide template 200 illustrated in FIG. 1 defines a plurality of additional curved channels 220 configured to optionally receive the medical instrument. The curved channel 204 and plurality of additional curved channels 220 together define a matrix 222 of curved channels.

For the purposes of describing possible relative positions of the various curved channels, the present disclosure defines various sets of curved channels that together form the matrix 222. Furthermore, for the sake of simplicity, when referring to all the curved channels in a set or all the curved channels within the matrix 222, the nomenclature “curved channels 204” will continue to be used, wherein each individual curved channel 204 within the set or matrix 222 continues to have a respective first opening 214 and second opening 216.

For instance, as shown in FIG. 1, a first set 224 of curved channels 204 of the matrix 222 is defined as the subset of curved channels 204 aligned across a width of the first face 206 and across a width of the third face 210 of the guide template 200. Each curved channel 204 within the set extends between a respective first opening 214 defined on the first face 206 and a second opening 216 defined on the third face 210. As further shown in FIG. 1, a second set 226 of curved channels 204 of the matrix 222 similar to the first set 224 are also aligned across a width of the first face 206 and across a width of the third face 210 of the guide template 200. As shown, the respective second openings 216 of the curved channels 204 of the second set 226 are disposed directly adjacent the second openings 216 of the curved channels 204 of the first set 224 along a length of the third face 210.

In some examples, including the illustrated implementation shown in FIG. 1, it is contemplated that this pattern can be extended across the first face 206 and third face 210 of the guide template 200. Specifically, as illustrated, a third set 228 of curved channels 204 of the matrix 222 are aligned across a width of the first face 206 and across a width of the third face 210 of the guide template 200, and the respective second openings 216 of the curved channels 204 of the third set 228 are disposed a first distance offset from the second openings 216 of the curved channels 204 of the second set 226.

Furthermore, as shown, a fourth set 230 of curved channels 204 of the matrix 222 are aligned across a width of the first face 206 and across a width of the third face 210 of the guide template 200, and the respective second openings 216 of the curved channels 204 of the fourth set 230 are disposed a second distance offset from the second openings 216 of the curved channels 204 of the third set 228 of curved channels 204, wherein the second distance is greater than the first distance.

Furthermore, as shown, a fifth set 232 of curved channels 204 of the matrix 222 are aligned across a width of the first face 206 and across a width of the third face 210 of the guide template 200, and the respective second openings 216 of the curved channels 204 of the fifth set 232 are disposed a third distance offset from the second openings 216 of the curved channels 204 of the fourth set 230 of curved channels 204, wherein the third distance is greater than the second distance.

Furthermore, as shown, a sixth set 234 of curved channels 204 of the matrix 222 are aligned across a width of the first face 206 and across a width of the third face 210 of the guide template 200, and the respective second openings 216 of the curved channels 204 of the sixth set 234 are disposed a fourth distance offset from the second openings 216 of the curved channels 204 of the fifth set 232 of curved channels 204, wherein the fourth distance is greater than the third distance.

Furthermore, as shown, a seventh set 236 of curved channels 204 of the matrix 222 are aligned across a width of the first face 206 and across a width of the third face 210 of the guide template 200, and the respective second openings 216 of the curved channels 204 of the seventh set 236 are disposed a fifth distance offset from the second openings 216 of the curved channels 204 of the sixth set 234 of curved channels 204, wherein the fifth distance is greater than the fourth distance.

Accordingly, when viewing the guide template 200 normal to the first face 206, the first openings 214 of the curved channels 204 in the matrix 222 are arranged generally equidistant from each other. Conversely, when viewing the guide template 200 normal to the third face 210, the second openings 216 of the curved channels 204 in the matrix 222 are arranged generally in sets spaced apart along the third face 210, where the distance between sets gradually increases along the third face 210.

In other words, the curved channels 204 in the first set 224 have a first radii of curvature. The curved channels 204 in the second set 226 have a second radii of curvature that are greater than the first radii of curvature. The curved channels 204 in the third set 228 have a third radii of curvature that are greater than the second radii of curvature. The curved channels 204 in the fourth set 230 have a fourth radii of curvature that are greater than the third radii of curvature. The curved channels 204 in the fifth set 232 have a fifth radii of curvature that are greater than the fourth radii of curvature. The curved channels 204 in the sixth set 234 have a sixth radii of curvature that are greater than the fifth radii of curvature. The curved channels 204 in the seventh set 236 have a seventh radii of curvature that are greater than the sixth radii of curvature.

Advantageously, the various curved channels 204 within the matrix 222 provide many alternative insertion paths through which a user can insert a medical instrument 150. This allows for meticulous control over the trajectory of the medical instrument 150 to ensure accurate placement at the target tissue of the patient. Furthermore, in some examples, the medical instrument 150 can include several elongated needle structures such that multiple curved channels 204 can be utilized at one time.

Additionally, it is understood that although each set 224, 226, 228, 230, 232, 234, 236 in the illustrated implementation includes 7 curved channels 204, other numbers of curved channels 204 are contemplated within each set. Further still, it is understood that in some implementations, some sets can have different numbers of curved channels 204 than other sets. Further still, although the matrix 222 in the illustrated implementation includes 7 sets of curved channels 204, it is understood that in some implementations, the matrix 222 can include a different number of sets of curved channels 204. For instance, a given matrix 222 can have a range of about 1 set to about 20 sets, such as about 5 sets to about 10 sets.

Furthermore, as shown in FIG. 1, the guide template 200 includes coupling means 218 for coupling the guide template 200 to an automated chassis 300, which is described in greater detail below. In the illustrated example, the coupling means 218 include clips that reversibly engage with complementary features on the automated chassis 300.

Referring to FIG. 2, the guide template 200 is shown within in conjunction with an automated chassis 300 that is configured to move the guide template 200. The automated chassis 300 includes an actuator 302 that provides propulsion to move the guide template 200 and a rail system 304 that defines a path along which the guide template 200 can travel. Accordingly, the guide template 200 is couplable with the automated chassis 300 such that the position of the guide template 200 can be controlled by the automated chassis 300. As shown in FIG. 1, the guide template 200 is coupled to the rail system 304 of the automated chassis 300, for example, via coupling means 218. By controlling the actuator 302, the position of the guide template 200 is movable along a length of the rail system 304.

In the illustrated example, the 304 is linear such that the path along which the guide template 200 can travel is also linear. However, it is contemplated herein that the rail system 304 could extend along a curved path so as to permit the guide template 200 to move along a curved path relative to the patient.

Furthermore, FIG. 2 includes an illustrative example of the guide template 200 shown in partial transparency. Accordingly, the matrix 222 of curved channels 204 are visible.

Together, the guide template 200 and the automated chassis 300 form a system 100 for positioning a medical instrument 150.

As provided herein, in some examples, including the implementation illustrated in FIG. 2, the system 100 is magnetic resonance (MR) compatible so as not to interfere with MR imaging. Accordingly, various components of the system, including the guide template 200 and elements of the automated chassis 300, such as the actuator 302, are made from MR compatible materials. For instance, in some implementations, the actuator 302 is a pneumatic actuator 302 with air-powered pneumatic motors.

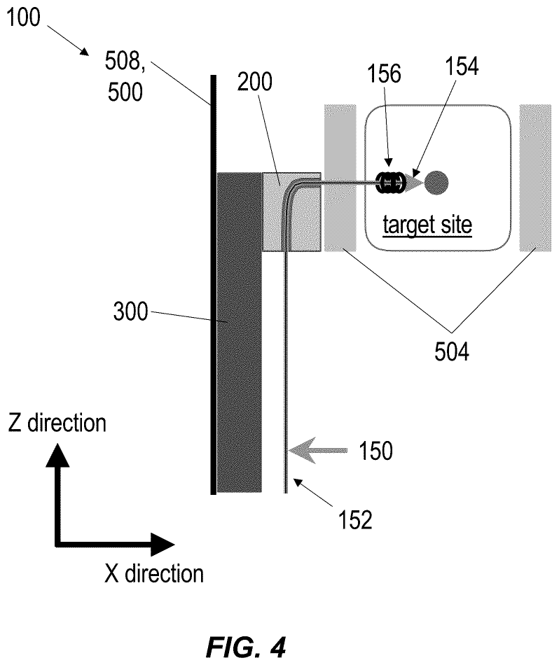

Advantageously, MR compatibility allows the system 100 to be implemented during intraoperative use of a magnetic resonance imaging (MRI) machine 500. FIG. 4 shows a top view of a schematic of an example system 100 in use with a medical instrument 150, in accordance with an illustrative embodiment.

Specifically, FIG. 4 shows the system 100 positioned within a MRI machine 500 such that the automated chassis 300 extends along a gantry wall 508 of the MRI machine 500 along the Z direction. As shown, the targeted tissue is located at a target site of the patient is positioned between two compression plates 504 that help stabilize the target tissue (for example, breast tissue). The guide template 200 and automated chassis 300 are thus positioned between the gantry wall 508 and a compression plate 504.

### Example Control System

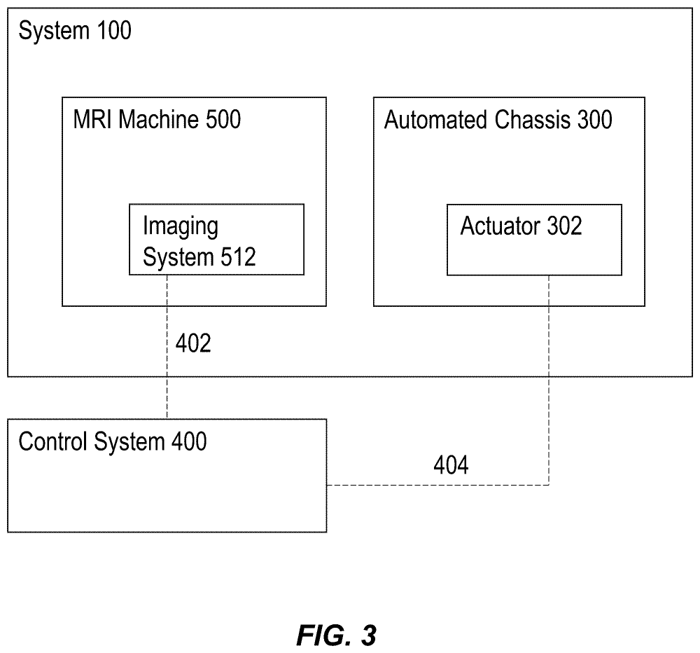

Referring to FIG. 3, an example system 100 with connections between various elements of the present disclosure including, for example, the actuator 302 of the automated chassis 300 of FIG. 2 is shown coupled to an example control system 400. As further described herein, the example system 100 in the illustrated implementation includes an MRI machine 500 (e.g., the MRI machine 500 of FIGS. 4-11) including an imaging system 512 coupled to the MRI machine 500. The imaging system 512 is configured to gather, interpret, and output data from the MRI machine 500. For example, the imaging system 512 is configured to output images of the patient and target tissue to an output device, a display 514, and/or a user interface. The device automated chassis 300 is disposed within the MRI machine 500, similar to the configurations shown in FIGS. 4-11.

The control system 400 is disposed adjacent to the MRI machine 500. The control system 400 is operatively coupled to and/or in communication with the imaging system 512 (e.g., to receive imaging data from the MRI machine 500), for instance, via connection line 402. The control system 400 is also operatively coupled to and/or in communication with the actuator 302 of the automated chassis 300 via a connection line 404. The various connection lines 402 and 404 can include a fiber optic cable or any other MRI-compatible data link.

Accordingly, the control system 400 is in electrical communication with the actuator 302 and is configured to modulate the actuator 302. For example, compressed air or another fluid can be delivered to a pneumatic motor of the actuator 302. As provided herein, the actuator 302 is coupled to the guide template 200. Accordingly, the control system 400 can selectively actuate the actuator 302 to facilitate translation of the guide template 200 along the rail system 304 toward a desired position, thereby moving a medical instrument 150 toward a target tissue of a patient (e.g., a biopsy needle toward a suspected lesion in a patient's breast).

In some examples, as further provided herein, the control system 400 can recursively operate to adjust the position of the guide template 200 and medical instrument 150 based on information from the MRI machine 500. Data from the imaging system 512 (or preconfigured instructions in the control system 400) can identify a target tissue of the patient with respect to the location of the distal end 154 of the medical instrument 150. Accordingly, the actuator 302 is configured to receive an output from the control system 400 to determine a target position of the guide template along the length of the rail system. In some implementations, the control system 400 is operatively coupled to a computing device 600 that includes a processing unit 606. The processing unit 606 can be operatively connected to the imaging system 512 of the MRI machine 500 so as to receive images from the MRI machine 500. These images correspond to the instant position of the medical instrument 150 and thus the guide template 200. The processing unit 606 can then provide a corresponding output to the control system 400 that is in turn configured to send an output to the actuator 302, thereby moving the guide template 200 as desired so that the instant position of the medical instrument 150 is moved toward the target position.

In some examples, localizing the distal end 154 of the medical instrument 150 is aided by the inclusion of a micro-tracking coil 156 embedded in the distal end 154 of the medical instrument 150. Then, the control system 400 can actuate the actuator 302 of the automated chassis 300 by selectively actuating pneumatic motors of the actuator 302, thereby adjusting the trajectory of the medical instrument 150. The imaging system 512 can be used to verify the trajectory of the medical instrument 150 and/or the position of the medical instrument 150 relative to the target tissue once inserted into the patient.

As further provided herein, as the medical instrument 150 is inserted into the patient, the MRI machine 500 continuously feeds data from the imaging system 512 to the control system 400. The control system 703 may include a user interface configured to display imaging data from the 512. Such a user interface can show the location of the medical instrument 150 within the patient. A clinician (or an automated system) can thus interpret the imaging data and adjust the insertion trajectory of the medical instrument 150.

In some implementations, the processing unit 606 of the computing device 600 can analyze the images received by the imaging system 512 using imaging processing techniques. For example, the images can be analyzed using an automated system that is configured identify the location of target tissues such as suspected cancerous lesions.

However, in some implementations, the imaging system 512 and the actuator 302 are not in electrical communication. Accordingly, a user may simply refer to images displayed on the display 514 and provide manual inputs to the control system 400, thereby manually adjusting the position of the guide template 200 and the medical instrument 150.

### Example Method

In one aspect, the techniques described herein relate to a method of positioning a medical instrument 150 in or relative to a patient. Although the system 100 described herein can be used to position any number of medical device medical instrument 150 relative to a desired target tissue of a patient, in the illustrated implementations shown in FIGS. 4-11, the target tissue is disposed in the breast of a patient. Accordingly, the medical instrument 150 used in conjunction with the system 100 is a biopsy needle. However, in further use cases, other medical instrument 150, especially other needle-based medical instruments 150, can be used.

As provided herein, a user is able to engage the actuator 302 of the automated chassis 300 (for instance, by operation of the control system 400) to move the guide template 200 along the rail system 304 (for example along the Z direction) to a first position relative to the patient and a medical instrument 150 can be advanced through a curved channel 204 defined in the guide template 200. In the example illustrated in FIG. 4, the curved channel 204 facilitates redirection of the medical instrument 150 from the Z direction, that does not intersect the patient, to the X direction, that does intersect the patient.

Specifically, in various use cases, a medical instrument 150 can be slidably moved within the curved channel 204. For instance, the medical instrument 150 can be in the form of an elongated body, such as a needle, extending between a proximal end 152 and a distal end 154. Accordingly, the distal end 154 of the needle can be inserted into the first opening 214 and advanced through the curved channel 204 (for example, by sliding) until the distal end 154 remerges from the second opening 216. As the medical instrument 150 is advanced through the curved channel 204, it is redirected in accordance with the curved direction defined by the curved channel 204 such that the medical instrument 150 may be fed into the first opening 214 of the guide template 200 from a first direction and exit the second opening 216 of the guide template 200 in a second direction along a trajectory skewed or diverted relative to the first direction. For instance, in the illustrated examples, the second direction is diverted about 90 degrees relative to the first direction. However, in further examples, the second direction can be diverted relative to the first direction by a range of about 10 degrees to about 135 degrees. In other words, the second direction can be diverted at any angle relative to the first direction so long as the medical instrument 150 is disposed to move toward the patient after passing through the curved channel 204.

MR imaging can then be obtained to determine the position of the distal end 154 of the medical instrument 150 relative to a target tissue of the patient. In view of this MR imaging, the user can further engage the actuator 302 to move the guide template 200 along the rail system 304 of the automated chassis 300 to a second position relative to the patient, thereby repositioning the distal end 154 of the medical instrument 150 relative to the target tissue of the patient. In some instances, a user may alternatively reposition the medical instrument 150 into another one of the curved channels 204 of the matrix 222 in order to alter the trajectory of the medical instrument 150.

In this manner, a user may recursively image the patient under MR and reposition (i.e., steer) the guide template 200 toward a desired position relative to the target tissue of the patient by operation of the system 100. In some instances, the user may control the position of the medical instrument 150 while obtaining MR images continuously (e.g., about 3-5 images per second). Advantageously, this facilitates real-time intraoperative MR guidance for the placement of the medical instrument 150 into the target tissue of a patient. Accordingly, the example system 100 can increase targeting accuracy and thereby reduce false negative rates.

In some instances, the system 100 can include a display 514 operatively coupled to the MRI machine in order to provide continuous images to the user. This display 514 can inform real-time control of the system 100 and positioning of the medical instrument 150 relative to the patient.

Additionally, the example medical instrument 150 shown in FIG. 4 includes a micro-tracking coil 156 positioned at the distal end 154. The micro-tracking coil 156 is imageable under MR such that it can be readily located. Thus, by integrating one or more micro-tracking coils 156 into the medical instrument 150 itself, the user is provided rapid and high-quality feedback about the position of the distal end 154 relative to the target tissue real-time during the procedure. In further implementations, the medical instrument 150 can also be localized using conventional T1 or T2-weighted MRI scanning.

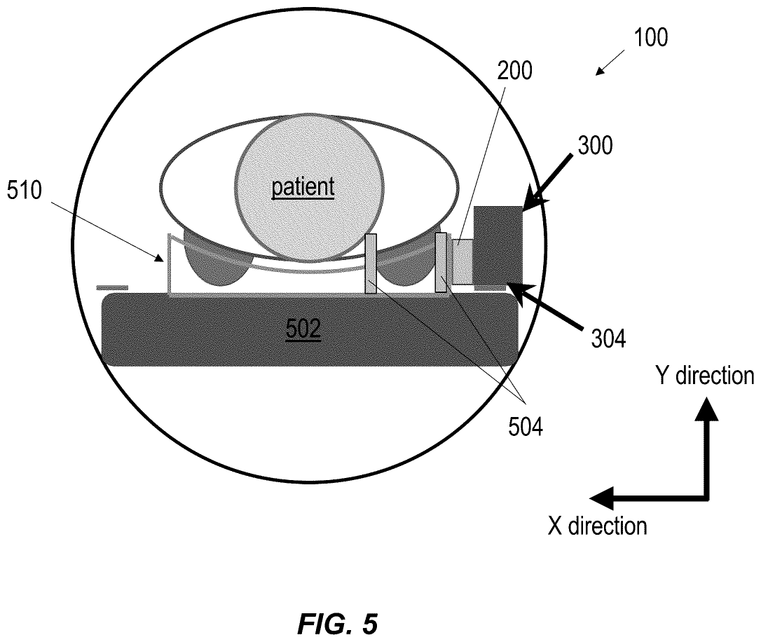

Referring to FIG. 5, an example system 100 is shown in which the guide template 200 and automated chassis 300 are positioned within an MRI machine as described with reference to FIG. 4. Furthermore, FIG. 5 shows a front view of the patient positioned within the MRI machine laying prone on a bed 502 such that the patient's breasts are positioned within a breast coil 510, which can improve the clarity of the MR images of the target tissue. As shown, the breast containing the target tissue is stabilized between the two compression plates 504. In some examples, as shown in FIG. 6, the breast coil 510 is contained within a support structure 506 that can help orient the patient and improve comfort. Accordingly, in use, the guide template 200 can be positioned such that the distal end 154 of the medical instrument 150 is positioned within the breast coil 510.

Referring to FIG. 6, a top view of the example system 100 of FIG. 5 is shown in use with a medical instrument 150. In FIG. 6, the guide template 200 is illustrated in partial transparency to show the trajectories of the curved channels 204 extending through the guide template 200. As shown, a medical instrument 150 is advanced through a curved channel 204 along the curved direction indicated by the arrow. Thus, the path of the medical instrument 150 is redirected along the curved channel 204 and toward the patient so that the distal end 154 of the medical instrument 150 is advanced toward a target tissue

As shown, the patient is positioned on a support structure 506 that is configured to support and orient the patient's body and define a region in which the target tissue can be isolated. Specifically, the support structure 506 includes a breast coil 510.

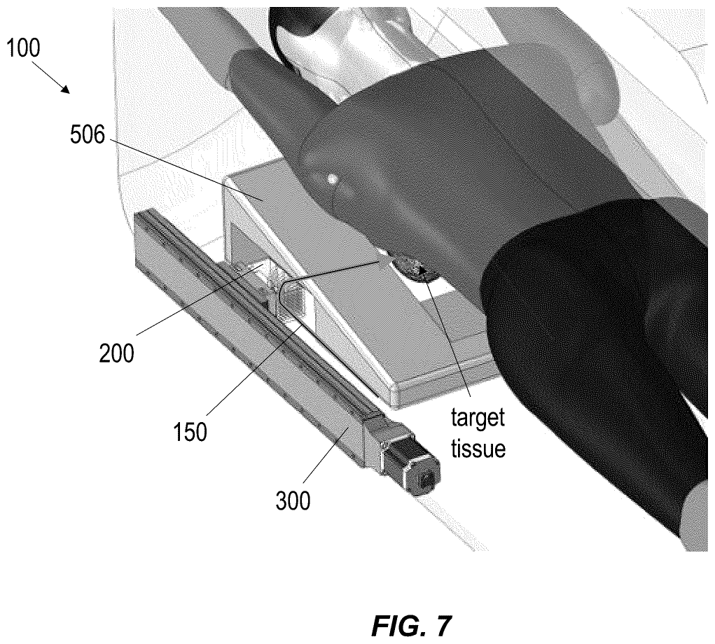

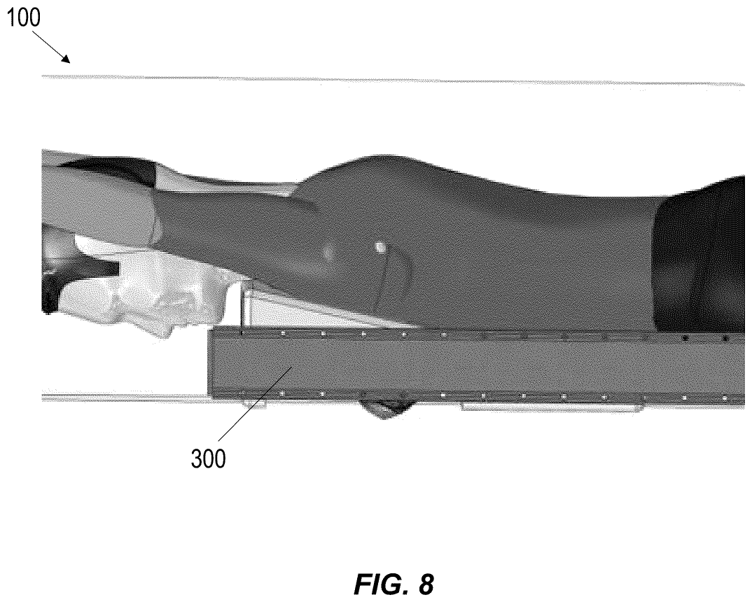

Referring to FIG. 7, a perspective view of the example system 100 of FIG. 6 is shown in use with the medical instrument 150. As shown, the guide template 200 is positioned along the rail system 304 of the automated chassis 300 such that one or more curved channels 204 are aligned with the target tissue of the patient. Accordingly, the 150 is directed through the curved channels 204 and toward the target tissue of the patient. As shown, the medical instrument 150 is advanced through a curved channel 204 along the curved direction indicated by the arrow. Furthermore, FIG. 8 shows a side view of the example system 100 of FIG. 6 positioned relative to the patient. As shown, the patient's body is supported by the support structure 506, which defines a region for the target tissue to be accessed by the medical instrument 150.

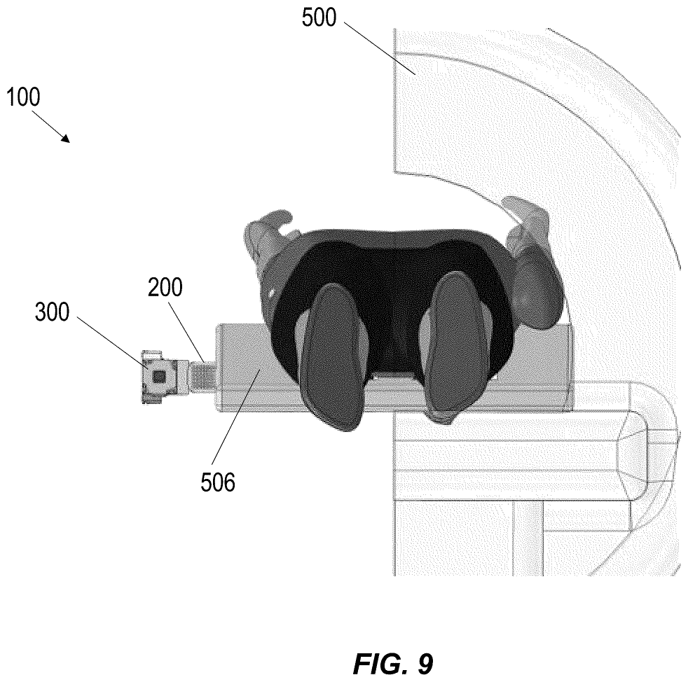

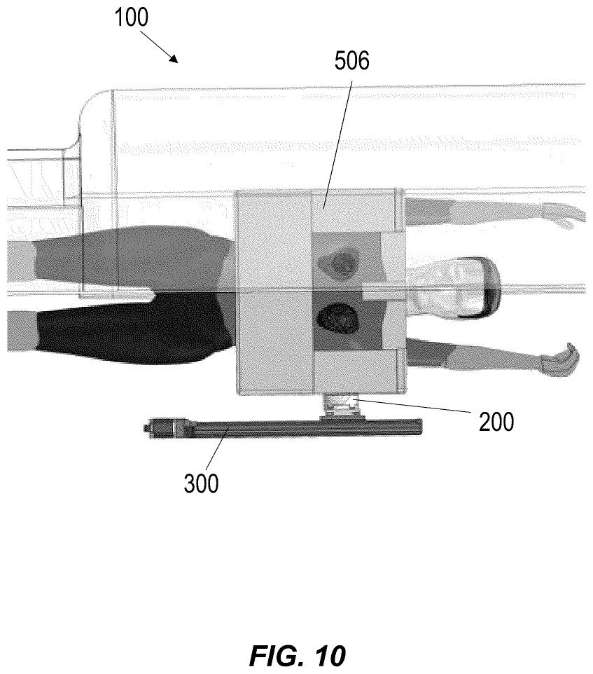

Referring to FIG. 9, a back view of the example system 100 of FIG. 6 is shown adjacent the patient within a MRI machine 500 (shown in cross-section). As shown, the patient is positioned on the support structure 506. The support structure 506 is in turn placed on a bed 502 (not shown) within the MRI machine 500. As provided herein, the system 100 can be sized and configured to be disposed alongside the patient within inner diameter of an MRI machine 500. However, in further examples, the MRI machine 500 can be integrated into the body of the MRI machine 500 itself. Furthermore, FIG. 10 shows a bottom view of the example system 100 of FIG. 6 positioned relative to the patient within the MRI machine 500. As shown, the automated chassis 300 is aligned axially parallel with the patient's body such that the rail system 304 extends parallel to a length of the patient's body. In some implementations, the length of the rail system 304 can be tailored to accommodate the scale of the anatomical region of interest. Furthermore, as provided herein, although the rail system 304 in FIG. 10 is shown to extend linearly, in further implementations, the rail system 304 can be curved to complement patient anatomy and/or to improve positional flexibility of the guide template 200 and thus the medical instrument 150.

Referring to FIG. 11, a bottom perspective view of the example system 100 of FIG. 6 positioned relative to a patient. With the guide template 200 illustrated in partial transparency, the curved channels 204 are shown extending through the 200 such that the second openings 216 of at least some of the curved channels 204 are aligned with the target tissue of the patient.

Advantageously, the methods provided herein can improve the workflows of various medical procedures. For instance, in the context of breast biopsies, conventional steps can include: (1) patient preparation; (2) preliminary MR imaging; (3) identification of the target tissue; (4) setup of a conventional template; (5) administration of local anesthesia; (6) insertion of an obturator; (7) additional MR imaging; (8) verification of obturator placement; (9) biopsy of the target tissue outside the MRI machine; (10) additional MR imaging to verify biopsy; and (11) confirmational MR imaging. The present systems, methods, and devices can enable several of these steps to be modified or even eliminated, including: (4) setup of a conventional template; (5) administration of local anesthesia; (6) insertion of an obturator; (7) additional MR imaging; (8) verification of obturator placement; (9) biopsy of the target tissue outside the MRI machine; and (10) additional MR imaging to verify biopsy.

Alternatively, or additionally, some implementations of the present disclosure can be configured to require minimum changes to existing accessories used for surgical procedures, reduce procedure time, and be compatible with existing navigation/planning systems. In some implementations, the systems, methods, and devices described herein can avoid having to remove a patient from the MRI machine before inserting the medical instrument 150, thereby shortening overall procedure time.

### Example Computing System

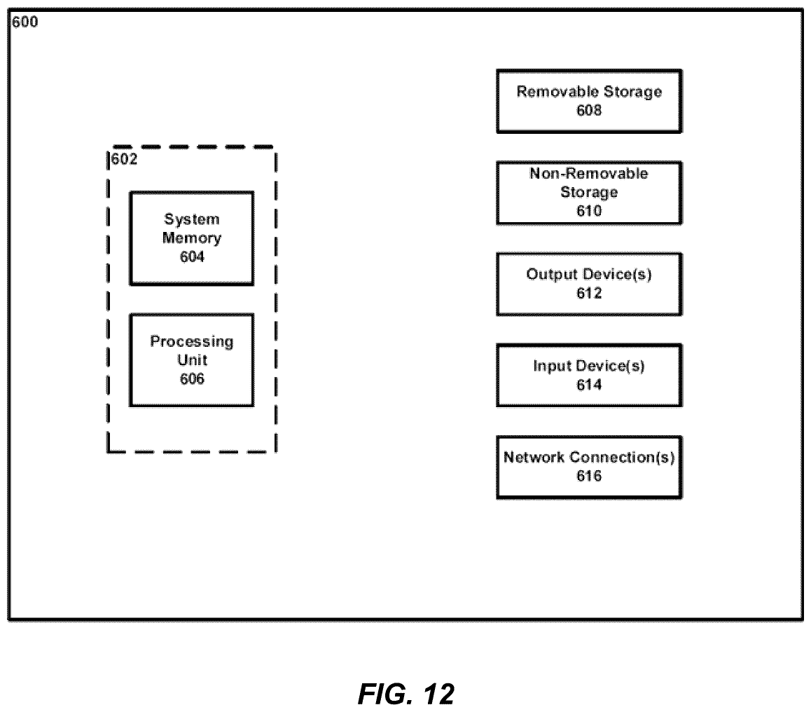

Referring to FIG. 12, an example computing device 600 upon which the methods described herein may be implemented is illustrated. It should be understood that the example computing device 600 is only one example of a suitable computing environment upon which the methods described herein may be implemented. Optionally, the computing device 600 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 600 typically includes at least one processing unit 606 and system memory 604. Depending on the exact configuration and type of computing device, system memory 604 may be volatile (such as random access memory (RAM)), non-volatile (such as read-only memory (ROM), flash memory, etc.), or some combination of the two. This most basic configuration is illustrated in FIG. 12 by dashed line 602. The processing unit 606 may be a standard programmable processor that performs arithmetic and logic operations necessary for the operation of the computing device 600. The computing device 600 may also include a bus or other communication mechanism for communicating information among various components of the computing device 600.

Computing device 600 may have additional features/functionality. For example, computing device 600 may include additional storage such as removable storage 608 and non-removable storage 410, including, but not limited to, magnetic or optical disks or tapes. Computing device 600 may also contain network connection(s) that allow the device 600 to communicate with other devices. Computing device 600 may also have input device(s) 614 such as a keyboard, mouse, touch screen, etc. Output device(s) 612, such as a display, speakers, printer, etc., may also be included. The additional devices may be connected to the bus in order to facilitate the communication of data among the components of the computing device 600. All these devices are well-known in the art and need not be discussed at length here.

The processing unit 606 may be configured to execute program code encoded in tangible, computer-readable media. Tangible, computer-readable media refers to any media that is capable of providing data that causes the computing device 600 (i.e., a machine) to operate in a particular fashion. Various computer-readable media may be utilized to provide instructions to the processing unit 606 for execution. Example of tangible, computer-readable media may include, but is not limited to, volatile media, non-volatile media, removable media, and non-removable media implemented in any method or technology for storage of information such as computer-readable instructions, data structures, program modules or other data. System memory 604, removable storage 608, and non-removable storage 610 are all examples of tangible, computer storage media. Examples of tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 606 may execute program code stored in the system memory 604. For example, the bus may carry data to the system memory 604, from which the processing unit 606 receives and executes instructions. The data received by the system memory 604 may optionally be stored on the removable storage 608 or the non-removable storage 410 before or after execution by the processing unit 606.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

### CONCLUSION

Various sizes and dimensions provided herein are merely examples. Other dimensions may be employed.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

What is claimed is:

1. A system for positioning a medical instrument, comprising:
a guide template having a body defining a curved channel extending through the guide template, the curved channel configured to receive the medical instrument,
an automated chassis comprising an actuator and a rail system,
wherein the guide template is coupled to the rail system of the automated chassis,
wherein a position of the guide template is movable along a length of the rail system, and
wherein the actuator moves the guide template along a length of the rail system.

2. The system of claim 1, further comprising a control system operatively coupled to the actuator,
wherein the actuator receives an output from the control system to determine a target position of the guide template along the length of the rail system.

3. The system of claim 1, wherein the body of the guide template comprises a first face, a second face opposite the first face, a third face, and a fourth face opposite the third face.

4. The system of claim 3, wherein the first face of the guide template defines a first opening and the third face of the guide template defines a second opening, and
wherein the curved channel extends through the body in a curved direction between the first opening and the second opening.

5. The system of claim 4, wherein the medical instrument comprises a proximal end and a distal end and is configured to slidably move within the curved channel.

6. The system of claim 3, wherein the fourth face of the guide template comprises coupling means for coupling the guide template to the rail system.

7. The system of claim 4, wherein the guide template defines a plurality of additional curved channels configured to receive the medical instrument, wherein the curved channel and plurality of additional curved channels together define a matrix of curved channels.

8. The system of claim 7, wherein a first set of curved channels of the matrix are aligned across a width of the third face of the guide template.

9. The system of claim 8, wherein a second set of curved channels of the matrix are aligned across a width of the third face of the guide template and are disposed directly adjacent the first set of curved channels along a length of the third face.

10. The system of claim 1, wherein the actuator comprises a magnetic resonance compatible actuator.

11. The system of claim 10, wherein the actuator is a pneumatic actuator.

12. The system of claim 1, wherein a distal end of the medical instrument comprises a micro-tracking coil configured to be located using a magnetic resonance imaging (MRI) machine.

13. The system of claim 1, wherein the medical instrument comprises a needle.

14. The system of claim 13, wherein the needle is a biopsy needle.

15. The system of claim 2, further comprising a magnetic resonance imaging (MRI) machine configured to continuously image the medical instrument.

16. The system of claim 15, further comprising a display operably coupled to the MRI machine and configured to display the position of the medical instrument to the user.

17. The system of claim 15, further comprising a processing unit operatively coupled to the MRI machine,
wherein the processing unit is configured to receive images from the MRI machine and provide a corresponding output to the control system; and
wherein the control system is configured to move the guide template toward the target position.

18. The system of claim 15 further comprising a breast coil.

19. The system of claim 18, wherein the target position of the guide template corresponds to a distal end of the medical instrument being positioned within the breast coil.

20. A method of positioning a medical instrument in a patient, the method comprising:
receiving images from a magnetic resonance imaging (MRI) machine;
moving a guide template along a length of a rail system of an automated chassis toward a target position adjacent the patient, wherein the guide template is coupled to a rail system of an automated chassis; and
advancing a medical instrument through a curved channel defined in the guide template.
